(19) United States
(12) Patent Application Publication
(10) Pub. No.: US 2009/0291113 A1
(43) Pub. Date: Nov. 26, 2009

(54) Osteogenic composition comprising a growth factor, a soluble cation salt and organic support

(75) Inventors: Remi Soula, Lyon (FR); Olivier Soula, Meyzieu (FR); Gerard Soula, Meyzieu (FR)

Correspondence Address: OLIFF & BERRIDGE, PLC, P.O. BOX 320850, ALEXANDRIA, VA, 22320-4850, US

(73) Assignee: ADOCIA, Lyon (FR)

(21) Appl. No.: 12/385,604

(22) Filed: Apr. 14, 2009

Related U.S. Application Data
(60) Provisional application No. 61/071,131, filed on Apr. 14, 2008.
(60) Provisional application No. 61/129,023, filed on May 30, 2008.
(60) Provisional application No. 61/129,617, filed on Jul. 8, 2008.
(60) Provisional application No. 61/193,217, filed on Nov. 6, 2008.

(30) Foreign Application Priority Data
Jul. 7, 2008 (FR) 0854618
Nov. 6, 2008 (FR) 0806222

(51) Int. Cl. A61K 38/18; A61F 2/00

(52) U.S. Cl. 424/423; 514/12

(57) ABSTRACT

An open implant, and a method for preparing the implant, constituted of an osteogenic composition with at least one osteogenic growth factor, one soluble salt of a cation at least divalent, and one organic support. The organic support has no demineralized bone matrix. In one embodiment, the implant is in the form of a lyophilizate.

The present invention relates to the field of osteogenic formulations, and more particularly formulations of osteogenic proteins belonging to the bone morphogenetic protein, BMP, family.

Bone morphogenetic proteins (BMPs) are growth factors involved in osteoinduction mechanisms. BMPs, also known as osteogenic proteins (OPs), were initially characterized by Urist in 1965 (Urist M R. Science 1965; 150, 893). These proteins, isolated from cortical bone, have the ability to induce bone formation in a large number of animals (Urist M R. Science 1965; 150, 893).

BMPs are expressed in the form of propeptides which, after post-translational maturation, have a length of between 104 and 139 residues. They possess great sequence homology with respect to one another and have similar three-dimensional structures. In particular, they have six cysteine residues involved in intramolecular disulfide bridges forming a “cysteine knot” (Scheufler C. 2004 J. Mol. Biol. 1999; 287, 103; Schlunegger M P, J. Mol. Biol. 1993; 231, 445). Some of them have a 7^{th} cysteine also involved in an intermolecular disulfide bridge responsible for the formation of the dimer (Scheufler C. 2004 J. Mol. Biol. 1999; 287:103).

In their active form, BMPs assemble as homodimers, or even as heterodimers, as has been described by Israel et al. (Israel D I, Growth Factors. 1996; 13(3-4), 291). Dimeric BMPs interact with BMPR transmembrane receptors (Mundy et al. Growth Factors, 2004, 22 (4), 233). This recognition is responsible for an intracellular signaling cascade involving, in particular, Smad proteins, thus resulting in target gene activation or repression.

BMPs, with the exception of BMPs 1 and 3, play a direct and indirect role on the differentiation of mesenchymal cells, causing differentiation of the latter into osteoblasts (Cheng H., J. Bone and Joint Surgery, 2003, 85A 1544-1552). They also have chemotaxis properties and induce proliferation and differentiation.

Some recombinant human BMPs, and in particular rhBMP-2 and rhBMP-7, have clearly shown an ability to induce bone formation in vivo in humans and have been approved for some medical uses. Thus, recombinant human BMP-2, dibotermin alfa according to the international nonproprietary name, is formulated in products sold under the name InFUSE® in the United States and InductOs® in Europe. This product is prescribed in the fusion of lumbar vertebrae and bone regeneration in the tibia for “nonunion” fractures. In the case of InFUSE® for the fusion of lumbar vertebrae, the surgical procedure consists, first of all, in soaking a collagen sponge with a solution of rhBMP-2, and then in placing the sponge in a hollow cage, LT cage, preimplanted between the vertebrae.

Recombinant human BMP-7, eptotermin alpha according to the international nonproprietary name, has the same therapeutic indications as BMP-2 and constitutes the basis of two products: OP-1 Implant for open fractures of the tibia and OP-1 Putty for the fusion of lumbar vertebrae. OP-1 Implant is composed of a powder containing rhBMP-7 and collagen, to be taken up in a 0.9% saline solution. The paste obtained is subsequently applied to the fracture during a surgical procedure. OP-1 Putty is in the form of two powders: one containing rhBMP-7 and collagen, the other containing carboxymethylcellulose (CMC). During a surgical procedure, the solution of CMC is reconstituted with a 0.9% saline solution and mixed with the rhBMP-7 and the collagen. The resulting paste is applied to the site to be treated.

Patent application US2008/014197 describes an osteoinductive implant constituted of a support (scaffold) containing a mineral ceramic, of a solid membrane integrally bonded to the support and of an osteogenic agent. The support is preferably a collagen sponge. The mineral ceramic comprises a calcium derivative, preferably a water-insoluble mineral matrix such as biphasic calcium phosphate ([0024], p 2). The solid membrane integrally bonded to the implant should be impermeable so as to limit the entry of cells from the surrounding soft tissues and also to prevent the entry of inflammatory cells ([0030], p 3). The entry of these cells into the implant is described as possibly resulting in a reduction in bone growth and in failure of the treatment ([0007], p 1).

This invention is centered on the addition of a membrane to the implant in order to improve osteogenesis.

Patent application US2007/0254041 describes a device in the form of a sheet containing a demineralized bone matrix (or DBM), particulate collagen and a physically crosslinked polysaccharide matrix. This implant may, moreover, contain an osteogenic substance such as a growth factor. The physically crosslinked polysaccharide acts as a stabilizing agent for the particles of demineralized bone ([0026], p 3), said alginate-based polysaccharide being crosslinked through the addition of calcium chloride.

Patent application WO96/39203 describes a biocompatible, osteogenic composite material with physical strength. This osteoinductive material is composed of demineralized bone, it being possible for the osteoinduction to take place only in the presence of demineralized bone, or in the presence of protein extracts of demineralized bone, or in the presence of these two elements according to the authors (lines 2-5, p 2). A calcium salt or a mineral salt is added to this material. The mineral salt is described as possibly being sodium hydroxide, sodium chloride, magnesium chloride or, magnesium hydroxide (lines 4-9, p 17). The calcium salt may or may not be a soluble salt (lines 20-21, p 17), and is preferably calcium hydroxide. The selection of the hydroxides of various cations, in particular calcium, to be added is justified by the effect of increasing the pH of the matrix, which favors increased collagen synthesis in this environment (lines 7-11, p 15).

This invention covers the formation of novel demineralized-bone-based implants, the physical and osteogenic properties of which would be improved by increasing the pH of the implant.

It has, moreover, been demonstrated that it is particularly advantageous to form complexes between a growth factor and a polymer with the aim of stabilizing it, of increasing its solubility and/or of increasing its activity.

It remains, however, essential to find a formulation which makes it possible to improve the effectiveness of these BMP growth factors in order to be able, for example, to reduce the amounts to be administered.

This problem is common to many growth factor formulations since these proteins are, in general, used at doses which exceed the physiological doses by several orders of magnitude.

It is to the applicant's credit to have found an osteogenic growth factor formulation which makes it possible to improve the activity of said growth factors through the addition of a solution of a soluble salt of a cation at least divalent, said soluble salt of a cation at least divalent potentiating the effect of the growth factor.

Surprisingly, this new formulation makes it possible to produce the same osteogenic effect with smaller amounts of growth factors.

The invention relates to an open implant constituted of an osteogenic composition comprising at least:
one osteogenic growth factor,
one soluble salt of a cation at least divalent, and
one organic support,
said organic support comprising no demineralized bone matrix.

The term “open implant” is intended to mean an implant which comprises neither a membrane nor a shell capable of limiting or regulating exchanges with the tissues surrounding the implant and which is substantially homogeneous in terms of the constitution thereof.

The term “demineralized bone matrix” (or DBM) is intended to mean a matrix obtained by acid extraction of autologous bone, resulting in loss of the majority of the mineralized components but in preservation of the collagen proteins or noncollagen proteins, including the growth factors. Such a demineralized matrix may also be prepared in inactive form after extraction with chaotropic agents.

The term “organic support” is intended to mean a support constituted of an organic matrix and/or a hydrogel.

The term “organic matrix” is intended to mean a matrix constituted of crosslinked hydrogels and/or collagen.

The organic matrix is a hydrogel obtained by chemical crosslinking of polymer chains. The interchain covalent bonds defining an organic matrix. The polymers that may be used for making up an organic matrix are described in the review by Hoffman, entitled Hydrogels for biomedical applications (Adv. Drug Deliv. Rev, 2002, 43, 3-12).

In one embodiment, the matrix is selected from matrices based on sterilized, preferably crosslinked, purified natural collagen.

The natural polymers such as collagen are extracellular matrix components which promote cell attachment, migration and differentiation. They have the advantage of being extremely biocompatible and are degraded by enzymatic digestion mechanisms. The collagen-based matrices are obtained from fibrillar collagen type I or IV, extracted from bovine or porcine tendon or bone. These collagens are first purified, before being crosslinked and then sterilized.

The organic supports according to the invention can be used as a mixture in order to obtain materials which may be in the form of a material with sufficient mechanical properties to be shaped or even molded, or else in the form of a “putty” where the collagen or a hydrogel plays a binder role.

Mixed materials can also be used, for example a matrix which combines collagen and inorganic particles and which may be in the form of a composite material with reinforced mechanical properties or else in the form of a “putty” or the collagen plays a binder role.

The inorganic materials that can be used comprise essentially ceramics based on calcium phosphate, such as hydroxyapatite (HA), tricalcium phosphate (TCP), biphasic calcium phosphate (BCP) or amorphous calcium phosphate (ACP), the main advantage of which is a chemical composition very close to that of bone. These materials have good mechanical properties and are immunologically inert. These materials may be in various forms, such as powders, granules or blocks. These materials have very different degradation rates, depending on their compositions; thus, hydroxyapatite degrades very slowly (several months) whereas tricalcium phosphate degrades more rapidly (several weeks). Biphasic calcium phosphates were developed for this purpose, since they have intermediate resorption rates. These inorganic materials are known to be principally osteoconductive.

The term “hydrogel” is intended to mean a hydrophilic three-dimensional network of polymer capable of adsorbing a large amount of water or of biological fluids (Peppas et al., Eur. J. Pharm. Biopharm. 2000, 50, 27-46). Such a hydrogel is constituted of physical interactions and is not therefore obtained by chemical crosslinking of the polymer chains.

Among these polymers may be found synthetic polymers and natural polymers. The polysaccharides forming hydrogels are described, for example, in the article entitled: Polysaccharide hydrogels for modified release formulations (Coviello et al. J. Control. Release, 2007, 119, 5-24).

In one embodiment, the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

In one embodiment, the polymer forming a hydrogel is selected from the group of natural polymers, among which are hyaluronic acid, keratan, pullulan, pectin, dextran, cellulose and cellulose derivatives, alginic acid, xanthan, carrageenan, chitosan, chondroitin, collagen, gelatin, polylysine and fibrin, and biologically acceptable salts thereof.

In one embodiment, the natural polymer is selected from the group of polysaccharides forming hydrogels, among which are hyaluronic acid, alginic acid, dextran, pectin, cellulose and its derivatives, pullulan, xanthan, carrageenan, chitosan and chondroitin, and biologically acceptable salts thereof.

In one embodiment, the natural polymer is selected from the group of polysaccharides forming hydrogels, among which are hyaluronic acid and alginic acid, and biologically acceptable salts thereof.

In one embodiment, said composition is in the form of a lyophilizate.

In one embodiment, the soluble salt of a cation at least divalent is a soluble salt of a divalent cation selected from calcium, magnesium or zinc cations.

In one embodiment, the soluble salt of a cation at least divalent is a calcium salt.

The term “soluble salt of a cation at least divalent” is intended to mean a salt of which the solubility is greater than or equal to 5 mg/ml, preferably 10 mg/ml, preferably 20 mg/ml.

In one embodiment, the soluble divalent-cation salt is a calcium salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is a magnesium salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is a zinc salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is calcium chloride.

In one embodiment, the soluble cation salt is a soluble multivalent-cation salt.

The term “multivalent cations” is intended to mean species carrying more than two positive charges, such as iron, aluminum, cationic polymers such as polylysine, spermine, protamine or fibrin.

The term “osteogenic growth factor”, or “BMP”, alone or in combination is intended to mean a BMP selected from the group of therapeutically active BMPs (bone morphogenetic proteins).

More particularly, the osteogenic proteins are selected from the group constituted of BMP-2 (dibotermin alpha), BMP-4, BMP-7 (eptotermin alpha), BMP-14 and GDF-5.

In one embodiment, the osteogenic protein is BMP-2 (dibotermin alpha).

In one embodiment, the osteogenic protein is GDF-5.

The BMPs used are recombinant human BMPs obtained according to the techniques known to those skilled in the art or purchased from suppliers such as, for example, the company Research Diagnostic Inc. (USA).

In one embodiment, the hydrogel may be prepared just before implantation.

In one embodiment, the hydrogel may be prepared and stored in a prefilled syringe in order to be subsequently implanted.

In one embodiment, the hydrogel may be prepared by rehydration of a lyophilizate just before implantation or may be implanted in dehydrated form.

Lyophilization is a water sublimation technique enabling dehydration of the composition. This technique is commonly used for the storage and stabilization of proteins.

The rehydration of a lyophilizate is very rapid and enables a ready-to-use formulation to be easily obtained, it being possible for said formulation to be rehydrated before implantation by the addition of blood, or implanted in its dehydrated form, the rehydration then taking place, after implantation, through the contact with the biological fluids.

In addition, it is possible to add other proteins, and in particular angiogenic growth factors such as PDGF, VEGF or FGF, to these osteogenic growth factors.

The invention therefore relates to a composition according to the invention, characterized in that it further comprises angiogenic growth factors selected from the group constituted of PDGF, VEGF or FGF.

The osteogenic compositions according to the invention are used by implantation, for example, for filling bone defects, for performing vertebral fusions or maxillofacial reconstructions, or for treating an absence of fracture consolidation (pseudarthrosis).

In these various therapeutic uses, the size of the matrix and the amount of osteogenic growth factor depend on the volume of the site to be filled.

In one embodiment, for a vertebral implant, the doses of osteogenic growth factor will be between 0.05 mg and 8 mg, preferably between 0.1 mg and 4 mg, more preferably between 0.1 mg and 2 mg, whereas the doses commonly accepted in the literature are between 8 and 12 mg.

In one embodiment, for a vertebral implant, the doses of angiogenic growth factor will be between 0.05 mg and 8 mg, preferably between 0.1 mg and 4 mg, more preferably between 0.1 mg and 2 mg.

As regards the uses in maxillofacial reconstruction or in the treatment of pseudarthrosis, for example, the doses administered will be less than 1 mg.

In one embodiment, the solutions of divalent cation have concentrations of between 0.01 and 1 M, preferably between 0.05 and 0.2 M.

In one embodiment, the solutions of anionic polysaccharide have concentrations of between 0.1 mg/ml and 100 mg/ml, preferably 1 mg/ml to 75 mg/ml, more preferably between 5 and 50 mg/ml.

The invention also relates to the method for preparing an implant according to the invention, which comprises at least the following steps:
a) providing a solution comprising an osteogenic growth factor,
b) providing an organic matrix and/or a hydrogel,
c) adding the solution containing the growth factor to the organic matrix and/or to the hydrogel, and optionally homogenizing the mixture,
d) adding a solution of a soluble salt of a cation at least divalent to the implant obtained in c),
e) optionally carrying out the lyophilization of the implant obtained in step d).

The invention also relates to the method for preparing an implant according to the invention, which comprises at least the following steps:
a) providing a solution comprising an osteogenic growth factor,
b) providing an organic matrix and/or a hydrogel,
c) adding a solution of a soluble salt of a cation at least divalent to the organic matrix and/or to the hydrogel b),
d) adding the solution containing the growth factor to the organic matrix and/or to the hydrogel obtained in c) and optionally homogenizing the mixture,
e) optionally carrying out the lyophilization of the implant obtained in step d).

In one embodiment, the organic matrix is a matrix constituted of crosslinked hydrogels and/or collagen.

In one embodiment, the matrix is selected from matrices based on sterilized, preferably crosslinked, purified natural collagen.

In one embodiment, in step c), the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

In one embodiment, in step b), the polymer forming a hydrogel, which may be crosslinked or noncrosslinked, is selected from the group of natural polymers, among which are hyaluronic acid, keratan, pectin, dextran, cellulose and cellulose derivatives, alginic acid, xanthan, carrageenan, chitosan, chondroitin, collagen, gelatin, polylysine and fibrin, and biologically acceptable salts thereof.

In one embodiment, in step b), the natural polymer is selected from the group of polysaccharides forming hydrogels, among which are hyaluronic acid, alginic acid, dextran, pectin, cellulose and its derivatives, pullulan, xanthan, carrageenan, chitosan and chondroitin, and biologically acceptable salts thereof.

In one embodiment, in step b), the natural polymer is selected from the group of polysaccharides forming hydrogels, among which are hyaluronic acid and alginic acid, and biologically acceptable salts thereof.

In one embodiment, in step c), the solution of a soluble salt of a cation at least divalent is a divalent-cation solution.

In one embodiment, the soluble divalent-cation salt is a calcium salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salt is calcium chloride.

In one embodiment, the soluble divalent-cation salts are magnesium salts, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, the soluble divalent-cation salts are zinc salts, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

In one embodiment, in step c), the solution of a soluble salt of a cation at least divalent is a multivalent-cation solution.

In one embodiment, the multivalent cations are selected from the group constituted of the multivalent cations of iron, aluminum, cationic polymers such as polylysine, spermine, protamine or fibrin.

In one embodiment, following step c), an organic matrix is impregnated with the formulation obtained in c) and then the addition of the solution of a cation at least divalent is carried out.

In one embodiment, in step a), a solution of a nonosteogenic growth factor is also provided.

The invention also relates to the use of the composition according to the invention, as a bone implant.

In one embodiment, said composition may be used in combination with a prosthetic device of the vertebral prosthesis or vertebral fusion cage type.

It also relates to the therapeutic and surgical methods using said composition in bone reconstruction.

The invention is illustrated by the following examples.

### EXAMPLE 1

### Preparation of Collagen Sponge/rhBMP-2 Implants

Implant 1: 40 μl of a solution of rhBMP-2 at 0.05 mg/ml are introduced sterilely into a Helistat type sterile 200 mm³ crosslinked collagen sponge (Integra LifeSciences, Plainsboro, N.J.). The solution is left to incubate for 30 minutes in the collagen sponge before use. The dose of rhBMP-2 is 2 μg.

Implant 2: It is prepared like implant 1, with 40 μl of a solution of rhBMP-2 at 0.5 mg/ml. The dose of rhBMP-2 is 20 μg.

### EXAMPLE 2

### Preparation of Implants of Collagen Sponge/rhBMP-2 with Calcium Chloride

Implant 3: 40 μl of a solution of rhBMP-2 at 1.5 mg/ml are introduced sterilely into a Helistat type sterile 200 mm³ crosslinked collagen sponge (Integra LifeSciences, Plainsboro, N.J.). The solution is left to incubate for 30 minutes in the collagen sponge before adding 100 μl of a solution of calcium chloride at a concentration of 18.3 mg/ml. After 15 minutes, the sponge is ready for use. The dose of rhBMP-2 is 20 μg.

### EXAMPLE 3

### Preparation of Implants of Collagen Sponge/rhBMP-2 with Calcium Chloride

Implant 4: 40 μl of a solution of rhBMP-2 at 0.15 mg/ml are introduced sterilely into a Helistat type sterile 200 mm³ crosslinked collagen sponge (Integra LifeSciences, Plainsboro, N.J.). The solution is left to incubate for 30 minutes in the collagen sponge before adding 100 μl of a solution of calcium chloride at a concentration of 18.3 mg/ml. The sponge is then subsequently frozen and lyophilized sterilely. The dose of rhBMP-2 is 2 μg.

Implant 5: It is prepared like implant 4, with 40 μl of a solution of rhBMP-2 at 1.5 mg/ml. The dose of rhBMP-2 is 20 μg.

### EXAMPLE 4

### Preparation of a Sodium Hyaluronate Gel Containing Calcium Chloride

Gel 1: 10.62 ml of sterile water are introduced into a 50 ml Falcon tube. 0.44 g of sodium hyaluronate (Pharma grade 80, Kibun Food. Chemifa, LTD) is added with vigorous stirring on a vortex. 0.14 g of calcium chloride is then added to the sodium hyaluronate gel, also with stirring. The concentration of calcium chloride in the gel is 13.1 mg/ml.

### EXAMPLE 5

### Preparation of a Sodium Hyaluronate Gel Containing rhBMP-2 and Calcium Chloride

Gel 2: 615 μl of a solution of rhBMP-2 at 0.57 mg/ml are prepared by diluting a solution of rhBMP-2 at 1.35 mg/ml in a buffer of Infuse type, with sterile water. This solution of rhBMP-2 is transferred into a sterile 10 ml syringe. 2.9 ml of the 4% sodium hyaluronate gel 1 containing calcium chloride at a concentration of 13.1 mg/ml are transferred into a sterile 10 ml syringe. The solution of rhBMP-2 is added to gel 1 by coupling the two syringes, and the gel obtained is homogenized by passing it from one syringe to the other several times. The final gel is transferred into a 10 ml Falcon tube. The concentration of rhBMP-2 in gel 2 is 0.10 mg/ml.

200 μl of gel 2 are injected per implantation site. The dose of rhBMP-2 implanted is 20 μg.

### EXAMPLE 6

### Evaluation of the Osteoinductive Capacity of the Various Formulations

The objective of this study is to demonstrate the osteoinductive capacity of the various formulations in a model of ectopic bone formation in the rat. Male rats weighing 150 to 250 g (Sprague Dawley OFA-SD, Charles River Laboratories France, B.P. 109, 69592 l'Arbresle) are used for this study.

An analgesic treatment (buprenorphine, Temgesic®, Pfizer, France) is administered before the surgical procedure. The rats are anesthetized by inhalation of an O₂-isoflurane mixture (1-4%). The fur is removed by shaving over a wide dorsal area. The skin of this dorsal area is disinfected with a solution of povidone-iodine (Vetedine® solution, Vetoquinol, France).

Paravertebral incisions of approximately 1 cm are made in order to free the right and left dorsal paravertebral muscles. Access to the muscles is made by transfascial incision. Each of the implants is placed in a pocket in such a way that no compression can be exerted thereon. Four implants are implanted per rat (two implants per site). The implant opening is then sutured using a polypropylene thread (Prolene 4/0, Ethicon, France). The skin is re-closed using a nonabsorbable suture. The rats are then returned to their respective cages and kept under observation during their recovery.

At 21 days, the animals are anesthetized with an injection of tiletamine-zolazepam (ZOLETIL® 25-50 mg/kg, IM, VIRBAC, France).

The animals are then sacrificed by euthanasia, by injecting a dose of pentobarbital (DOLETHAL®, VETOQUINOL, France). A macroscopic observation of each site is then carried out; any sign of local intolerance (inflammation, necrosis, hemorrhage) and the presence of bone and/or cartilage tissue are recorded and graded according to the following scale: 0: absence, 1: weak, 2: moderate, 3: marked, 4: substantial.

Each of the implants is removed from its implantation site and macroscopic photographs are taken. The size and the weight of the implants are then determined. Each implant is then stored in a buffered 10% formol solution.

Results:

This in vivo experiment makes it possible to measure the osteoinductive effect of rhBMP-2 by placing the implant in a muscle on the back of a rat. This non-bone site is termed ectopic.

The macroscopic observations of the explants enable us to evaluate the presence of bone tissues and to determine the mass of the explants.

| | | |
|---|---|---|
| Implant | Presence of bone tissues | Mass of explants (mg) |

| | |
|---|---|
| Implant 1 | Implants not found |

| | | |
|---|---|---|
| Implant 2 | 3.6 | 38 |
| Implant 3 | | 75 |
| Implant 4 | 3.2 | 26 |
| Implant 5 | 3.3 | 171 |
| Gel 2 | 3.7 | 122 |

A dose of 2 μg of rhBMP-2 in a collagen sponge (implant 1) does not have a sufficient osteoinductive capacity for it to be possible to find collagen implants after 21 days.

A dose of 20 μg of rhBMP-2 in a collagen sponge (implant 2) does not have a sufficient osteoinductive capacity for obtaining ossified implants with an average weight of 38 mg, after 21 days.

For the same dose of rhBMP-2 of 20 μg, the addition of calcium salts to the collagen sponge containing the rhBMP-2 makes it possible to increase the osteogenic activity of the rhBMP-2. The average mass of the ossified implants 3 is twice that of the implants 2.

Also for the dose of rhBMP-2 of 20 μg, lyophilization makes it possible to increase the effect of the calcium salt on the osteogenic activity of the rhBMP-2 (implant 5). The average mass of the lyophilized implants containing rhBMP-2 and the CaCl₂ is approximately four times greater than that of the implants containing only rhBMP-2 (implant 2). In addition, the bone score is equivalent between these implants.

For a dose of rhBMP-2 of 2 μg, rhBMP-2 in the presence of CaCl₂ lyophilized in the collagen sponge (implant 4) makes it possible to generate ossified implants, unlike rhBMP-2 alone at the same dose.

For a dose of rhBMP-2 of 20 μg, the sodium hyaluronate gel containing rhBMP-2 (gel 2) in the presence of calcium chloride makes it possible to increase the osteogenic activity of the rhBMP-2. The average mass of the explants obtained with gel 2 is approximately 3 times greater than that of the explants obtained with the collagen implants containing 20 μg of rhBMP-2 alone (implant 2).

1. An open implant constituted of an osteogenic composition comprising at least:
one osteogenic growth factor,
one soluble salt of a cation at least divalent, and
one organic support,
said organic support comprising no demineralized bone matrix.

2. The implant according to claim 1, wherein the support is constituted of an organic matrix and/or a polymer forming a hydrogel.

3. The implant according to claim 1, wherein the organic matrix is a matrix constituted of crosslinked hydrogels and/or collagen.

4. The implant according to claim 1, wherein the matrix is selected from matrices based on sterilized and crosslinked, purified natural collagen.

5. The implant according to claim 1, wherein the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

6. The implant according to claim 1, wherein the polymer forming a hydrogel, which may be crosslinked or noncrosslinked, is selected from the group of natural polymers, among which are hyaluronic acid, keratan, pullulan, pectin, dextran, cellulose and cellulose derivatives, alginic acid, xanthan, carrageenan, chitosan, chondroitin, collagen, gelatin, polylysine and fibrin, and biologically acceptable salts thereof.

7. The implant according to claim 6, wherein the natural polymer is selected from the group of polysaccharides forming hydrogels, among which are hyaluronic acid, alginic acid, dextran, pullulan, pectin, cellulose and its derivatives, xanthan, carrageenan, chitosan and chondroitin, and biologically acceptable salts thereof.

8. The implant according to claim 6, wherein the natural polymer is selected from the group of polysaccharides forming hydrogels, among which are hyaluronic acid and alginic acid, and biologically acceptable salts thereof.

9. The implant according to claim 1, wherein said composition is in the form of a lyophilizate.

10. The implant according to claim 1, wherein the osteogenic growth factor is selected from the group of therapeutically active BMPs (bone morphogenetic proteins).

11. The implant according to claim 1, wherein the osteogenic growth factor is selected from the group constituted of BMP-2 (dibotermin alpha), BMP-4, BMP-7 (eptotermin alpha), BMP-14 and GDF-5.

12. The implant according to claim 1, wherein the osteogenic protein is BMP-2 (dibotermin alpha).

13. The implant according to claim 1, wherein the osteogenic protein is GDF-5.

14. The implant according to claim 1, wherein it further comprises angiogenic growth factors selected from the group constituted of PDGF, VEGF or FGF.

15. The implant according to claim 1, wherein the a cation at least divalent is a divalent cation selected from the group constituted of calcium, magnesium or zinc salts.

16. The implant according to claim 1, wherein the soluble divalent-cation salt is a calcium salt, the counterion of which is selected from the chloride, the D-gluconate, the formate, the D-saccharate, the acetate, the L-lactate, the glutamate, the aspartate, the propionate, the fumarate, the sorbate, the bicarbonate, the bromide or the ascorbate.

17. The implant according to claim 1, wherein the soluble divalent-cation salt is calcium chloride.

18. The implant according to claim 1, wherein the a cation at least divalent is a multivalent cation selected from the group constituted of the cations of iron, aluminum or cationic polymers selected from polylysine, spermine, protamine and fibrin, alone or in combination.

19. A method for preparing an implant according to the invention, which comprises at least the following steps:
a) providing a solution comprising an osteogenic growth factor,
b) providing an organic matrix and/or a polymer forming a hydrogel,
c) adding the solution containing the growth factor to the organic matrix and/or to the hydrogel, and optionally homogenizing the mixture,
d) adding a solution of a soluble salt of a cation at least divalent to the implant obtained in c),
optionally carrying out the lyophilization of the implant obtained in step d).

20. The method according to claim 19, wherein the organic matrix is a matrix constituted of a crosslinked hydrogel and/or collagen.

21. The method according to claim 19, wherein the matrix is selected from matrices based on sterilized, crosslinked, purified natural collagen.

22. The method according to claim 20, wherein the polymer forming a hydrogel, which may be crosslinked or noncrosslinked, is selected from the group of synthetic polymers, among which are ethylene glycol/lactic acid copolymers, ethylene glycol/glycolic acid copolymers, poly(N-vinylpyrrolidone), polyvinylic acids, polyacrylamides and polyacrylic acids.

23. The method according to claim 20, wherein the polymer forming a hydrogel, which may be crosslinked or noncrosslinked, is selected from the group of natural polymers, among which are hyaluronic acid, keratan, pectin, dextran, cellulose and cellulose derivatives, alginic acid, xanthan, carrageenan, chitosan, chondroitin, collagen, gelatin, polylysine and fibrin, and biologically acceptable salts thereof.

24. The method according to claim 24, wherein, in step b), the natural polymer is selected from the group of polysaccharides forming hydrogels, constituted of hyaluronic acid, alginic acid, dextran, pectin, cellulose and its derivatives, pullulan, xanthan, carrageenan, chitosan and chondroitin, and biologically acceptable salts thereof.

25. The method according to claim 24, wherein the natural polymer is selected from the group of polysaccharides forming hydrogels, constituted of hyaluronic acid and alginic acid, and biologically acceptable salts thereof.

26. The method according to claim 20, wherein the solution of a soluble salt of a cation at least divalent is a divalent-cation solution.

27. The method according to claim 26, wherein, in step d), the soluble divalent-cation salt is selected from magnesium salts, the counterion of which is the chloride, the D-gluconate, the formate, the D-saccharate, the acetate, the L-lactate, the glutamate, the aspartate, the propionate, the fumarate, the sorbate, the bicarbonate, the bromide or the ascorbate.

28. The method according to claim 27, wherein, in step d), the soluble divalent-cation salt is selected from calcium salts, the counterion of which is the chloride, the D-gluconate, the formate, the D-saccharate, the acetate, the L-lactate, the glutamate, the aspartate, the propionate, the fumarate, the sorbate, the bicarbonate, the bromide or the ascorbate.

29. The method according to claim 27, wherein, in step d), the soluble divalent-cation salt is calcium chloride.

30. The method according to claim 20, wherein, in step a), a solution of a nonosteogenic growth factor is also provided.
